(19) United States
(12) Patent Application Publication
(10) Pub. No.: US 2006/0129148 A1
(43) Pub. Date: Jun. 15, 2006

(54) Screw sleeve made of polyetheretherketone (PEEK) for augmentation of bone screw insertion in osteoporotic or revision lumbar spine instrumentation

(76) Inventors: Edward D. Simmons, Williamsville, NY (US); Yinggang Zheng, Snyder, NY (US)

Correspondence Address: HODGSON RUSS LLP, ONE M & T PLAZA, SUITE 2000, BUFFALO, NY, 14203-2391, US

(21) Appl. No.: 11/242,624

(22) Filed: Oct. 3, 2005

Related U.S. Application Data
(60) Provisional application No. 60/615,431, filed on Oct. 1, 2004.

(51) Int. Cl. A61B 17/58

(52) U.S. Cl. 606/61

(57) ABSTRACT

An orthopaedic device comprising an orthopaedic bone screw and a sleeve of polyetheretherketone material in which the orthopaedic bone screw is received for increasing interference between the screw and bone tissue to increase strength of fixation when the device is installed in bone.

### CROSS REFERENCE TO A RELATED APPLICATION

Applicants claim priority based on Provisional Application No. 60/615,431 filed Oct. 1, 2004 and entitled “Screw Sleeve Made Of Polyetheretherketone (PEEK) For Augmentation Of Pedicle Screw Insertion In Osteoporotic Or Revision Lumbar Spine Instrumentation” which is incorporated herein by reference.

### BACKGROUND OF THE INVENTION

Pedicle screw instrumentation has been commonly used for thoracolumbar fixation. In osteoporotic spine with weak bone quality, loosening of pedicle screw anchoring is a major concern. High failure rates of pedicle screw fixation have been reported in the osteoporotic bone associated with poor fixation. In revision surgery with prior lumbar pedicle screw fixation, reinsertion of pedicle screw in the same pedicle hole, even though using a larger diameter screw, often has unsatisfactory anchoring force. Furthermore, the diameter of vertebral pedicle is anatomically the same, using oversize diameter screw may break through the pedicle wall. Several techniques or pedicle screw designs have been suggested to decrease the loosening of pedicle screw. Augmentation of pedicle screw with polymethylmethacrylate (PMMA) has been shown to improve pull out strength. Other studies suggested using expanded alligator screw to increase insertional torque, and improve pull out strength. PMMA is not an ideal material for bone to grow in and its complication with pulmonary embolism in vertebroplasty as well as the heat generated to endanger the surrounding bone are the major limitations for clinical use. Bioactive bone cement has been recommended with pilot studies but has not been commonly used. Studying for new materials and designs to improve the pedicle screw fixation is still evolving.

Polyetheretherketone (PEEK), commercially available from Victrex PLC of the United Kingdom under the designation PEEK-OPTIMA®, is a new medical grade polymer and is licensed by FDA Device and Drug Master Files for use as a medical implant material. The use of PEEK as an orthopaedic device material has increased in popularity in recent years, including artificial hip and knees, and spine fusion devices. PEEK has a similar modulus of elasticity to cortical bone. This particular stiffness encourages load sharing between implant material and nature bone, thereby stimulating bone healing effect. In vitro testing has shown that the content of osteoblastic proteins and the level of osteocalcin increases in the presence of PEEK contributing to increased bone healing. In addition, with high resistance to shape deformity, PEEK can help maintain an implant's shape over its service life. Lab investigations have shown that the presence of PEEK contributes to increased alkaline phosphate activity, leading to the formation of rapidly multiplying osteoplast-type cells. All these unique characteristics have made PEEK become an excellent biomaterial. Currently intervertebral cage is available for the interbody fusion.

### SUMMARY OF THE INVENTION

The excellent mechanical characteristics of polyetheretherketone, in particvular PEEK-OPTIMA®, allow it to be shaped into thin-walled designs. We propose to use PEEK-OPTIMA® to make a sleeve shape implant in conjunct with bone screw, such as pedicle screw, to improve the pull out strength and resistance to the toggling force which is produced during daily activities of low back. In osteoporotic bone or even in normal bone where strong anchoring forces are needed, screw sleeve will increase the interference between the screw and surrounding bone tissue therefore to significantly increase the strength of fixation.

In addition, we believe that this type of screw sleeve made of polyetheretherketone, in particular PEEK-OPTIMA®, can also be used for augmentation of screw anchoring in different orthopaedic procedures, such as internal fixation of fractures including, for example hip fracture and tibia fracture, in osteoporotic patients or other revision orthopaedic surgeries which require screw instrumentation. In other words, the invention can be applied to any bone screw, such as used in fracture fixation throughout the body, not just the spine.

The following detailed description of the invention, when read in conjunction with the accompanying drawings wherein the same reference numerals denote the same or similar parts throughout the several views, is in such full, clear, concise and exact terms as to enable any person skilled in the art to which it pertains, or with which it is mostly nearly connected, to make and use the invention.

### BRIEF DESCRIPTION OF THE DRAWINGS

FIG. 1 is an elevational view of a pedicle or bone screw for thoracolumbar fixation;

FIG. 2 is an elevational view of a screw sleeve according to the invention for use with the pedicle or bone screw of FIG. 1; and

FIG. 3 is an elevational view of a screw sleeve according to another embodiment of the invention.

### DETAILED DESCRIPTION

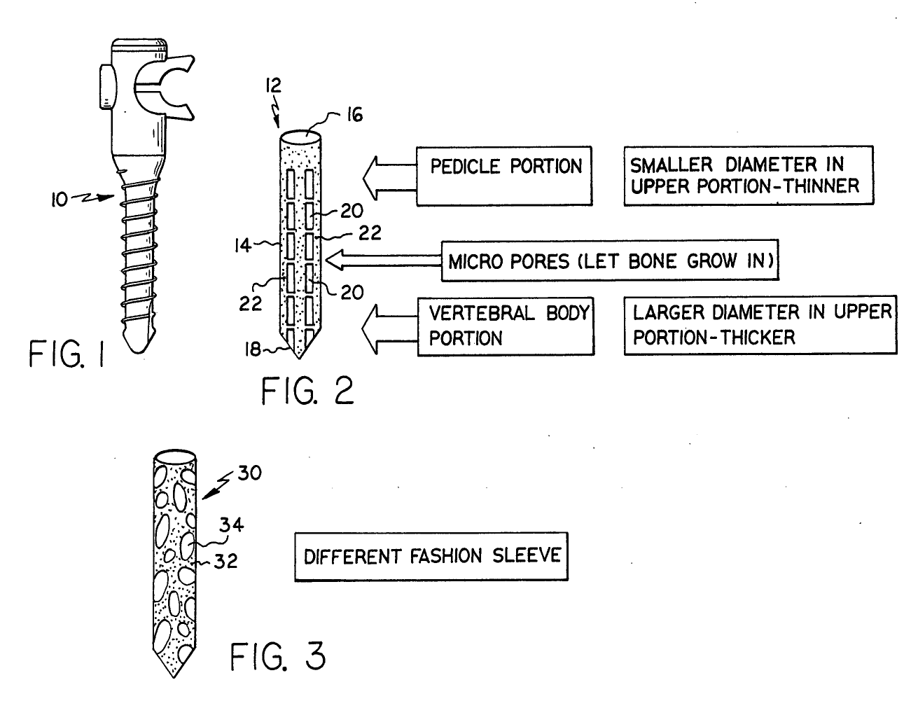

A pedicle screw 10 for thoracolumbar fixation is shown in FIG. 1. The same representation/diagram can be applied to any bone screw fixation, such as in fractures. A sleeve 12 of polyetheretherketone (PEEK) material, preferable PEEK-OPTIMA® is shown in FIG. 2. Sleeve 12 has a substantially cylindrical body portion 14 which is open at one end 16 and is closed in a tapered or conical portion 18 at the opposite end. The inner diameter of body portion 14 is of a dimension allowing it to receive a pedicle screw, like screw 10 of FIG. 1, therein. Body 14 has both openings 20 and micropores 22 to receive bone growth. In this embodiment the openings 20 are generally rectangular in shape.

Sleeve 30 in the embodiment of FIG. 3 similarly has micropores 32 in the body thereof and has openings 34 of somewhat irregular shapes.

Indications:
(1) Osteoporotic spine
(2) Revision surgery of the lumbar spine instrumentation. Taking out previous pedicle screw, re-inserting bigger diameter screw in the same pedicle hole with PEEK-OPTIMA® sleeve.

Anticipated advantages:
(1) Significantly improving pull out strength of pedicle screw following insertion simultaneously, thereby to prevent screw loosening.
(2) Absorbing the togging force sustained by screw.

It is therefore apparent that the invention accomplishes its intended objectives. While embodiments of the invention have been described in detail, that is done for the purpose of illustration, not limitation.

### REFERENCES

1. Invibio (2001); PEEK-OPTIMA, Technical information for the Medical Profession.
2. Poloneni, V. K. Wang, A. Essner, A., Un, R., Chopra, A., Stark, C., Dumbleton, J. H. (1998): Characterization of carbon fiber-reinforced PEEK composite for use as a bearing material in total hip replacements. ASTM Spec. Tech. Publ., STP 1346: 266-273.
3. Williams, D. F., McNamara, A. (1987): Potential of Polyarletherketone (PEEK) and carbon fiber reinforced PEEK in medical applications. J. Mater. Science Letters, 6: 188-190.
4. Morrison, C. Macnair, R., MacDonald, C., Wykman, A., Goldie, L., Grant, M. H. (1995): In vitro biocompatibililty testing of polymers for orthopaedic implants using cultured fibroplasts and osteoblasts. Biomaterials, 16(13): 987-982.

1. An orthopaedic device comprising:
a) an orthodaedic bone screw; and
b) a sleeve of polyetheretherketone material in which said screw is received for increasing interference between said screw and bone tissue when said device is installed in bone.

2. The orthopaedic device according to claim 1, wherein said sleeve is substantially cylindrical and open at one end and closed at an opposite end.

3. The orthopaedic device according to claim 2, wherein the opposite end is closed in a substantially conical portion.

4. The orthopaedic device according to claim 1, wherein the sleeve has openings therein to receive bone growth.

5. The orthopaedic device according to claim 4, wherein the openings are generally rectangular in shape.

6. The orthopaedic device according to claim 4, wherein the openings are irregular in shape.

7. The orthopaedic device according to claim 1, wherein said orthopaedic bone screw is a pedicle screw.

8. An orthopaedic device comprising a sleeve of polyetheretherketone material for receiving an orthopaedic bone screw therein, said sleeve being thin-walled and substantially cylindrical shape having an inner diameter of a size allowing an orthopaedic bone screw to be received in close-fitting relation therein, said sleeve being open at one end and closed in a tapered shape at an opposite end, said sleeve increasing interference between the othopaedic bone screw and bone tissue when installed in bone so as to increase strength of fixation of the screw in bone.

9. The orthopaedic device according to claim 8, wherein said sleeve has openings in the wall thereof to receive bone growth.

10. The orthopaedic device according to claim 9, wherein the openings are generally rectangular in shape.

11. The orthopaedic device according to claim 9, wherein the openings are irregular in shape.

12. The orthopaedic device according to claim 8, wherein the sleeve inner diameter is dimensioned to receive a pedicle screw.

13. A method of making an orthopaedic device comprising:
a) providing an orthopaedic bone screw;
b) forming a sleeve of polyetheretherketone material having an inner dimension allowing the orthopaedic bone screw to be received therein and having an open end and a closed end of tapering shape; and
c) inserting the orthopaedic bone screw into the sleeve from the open end toward the closed end;
d) so that when the orthopaedic device is installed in bone the sleeve increases interference between the orthopaedic bone screw and bone tissue.

14. The method according to claim 13, wherein the sleeve is formed in a generally thin-walled cylindrical shape.

15. The method according to claim 13 further including forming openings in the sleeve to receive bone growth.

16. The method according to claim 15, wherein the openings are formed in generally rectangular shapes.

17. The method according to claim 15, wherein the openings are formed in irregular shapes.
